(19) United States
(12) Patent Application Publication
(10) Pub. No.: US 2024/0378623 A1
(43) Pub. Date: Nov. 14, 2024

(54) USER INTERFACE AND ARCHITECTURE TO MITIGATE VEHICLE CARBON EMISSION

(71) Applicant: Rivian IP Holdings, LLC, Plymouth, MI (US)

(72) Inventors: Andrew Samuel Peterman, Pacifica, CA (US); Christopher Brown, Costa Mesa, CA (US); Nicholas Joseph Santero, Santa Cruz, CA (US); Brian Frederick Wang, San Francisco, CA (US)

(21) Appl. No.: 18/316,980

(22) Filed: May 12, 2023

(51) Int. Cl. G06Q 30/018

(52) U.S. Cl. CPC G06Q 30/018

(57) ABSTRACT

Aspects of this technical solution can determine, based on power applied to a vehicle from a source of power and power consumed by the vehicle, an emission metric corresponding to the vehicle, and send a request to a second system, the request to link with an asset based on the emission metric.

### INTRODUCTION

Individuals are increasingly interested in offsetting emissions and environmental impact from use of electricity derived from fossil-fuel sources, based on their particular usage. However, the varying conditions and circumstances around electricity usage and carbon emission with respect to individual use cases is prohibitive for an individual to perform manually.

### SUMMARY

This technical solution is directed to determining carbon emissions associated with activity of a particular vehicle over a particular period of time, and executing instructions to counteract the carbon emissions. For example, activity of a particular vehicle can correspond to a trip from a first origin location to a second destination location. A trip can include traversal of a distance between the origin and the destination by one or more roads there between. For example, a particular period of time can correspond to a time required to complete the trip, or an entire useful lifetime of a vehicle. The technical solution can determine carbon emissions based on factors associated with the trip, consumption of electricity by the vehicle, the carbon emissions associated with the power grid at which the vehicle has charged or may charge, and manufacture of the vehicle, or any combination thereof. For example, the technical solution can determine a carbon emission associated with manufacture of a particular vehicle. According to a determination of carbon emission, the technical solution can provide via a user interface of the vehicle or a device linked with the vehicle, a presentation to execute instructions to offset the determined amount of carbon emitted. For example, the user interface can cause a communication with a system to transfer or purchase carbon credits corresponding to the determined amount of carbon emitted.

At least one aspect is directed to a system. The system can include one or more processors coupled with a non-transitory memory. The system can determine, based on power applied to a vehicle from a source of power and power consumed by the vehicle, an emission metric corresponding to the vehicle. The system can send a request to a second system, the request to link with an asset based on the emission metric.

At least one aspect is directed to a method. The method can include determining, based on power applied to a vehicle from a source of power and power consumed by the vehicle, an emission metric corresponding to the vehicle. The method can include sending a request to a second system, the request to link with an asset based on the emission metric.

At least one aspect is directed to a non-transitory computer readable medium. The computer readable medium can include one or more instructions stored thereon and executable by a processor. The processor can determine, based on power applied to a vehicle from a source of power and power consumed by the vehicle, an emission metric corresponding to the vehicle. The processor can send a request to a second system, the request to link with an asset based on the emission metric.

These and other aspects and implementations are discussed in detail below. The foregoing information and the following detailed description include illustrative examples of various aspects and implementations, and provide an overview or framework for understanding the nature and character of the claimed aspects and implementations. The drawings provide illustration and a further understanding of the various aspects and implementations, and are incorporated in and constitute a part of this specification. The foregoing information and the following detailed description and drawings include illustrative examples and should not be considered as limiting.

### BRIEF DESCRIPTION OF THE DRAWINGS

The accompanying drawings are not intended to be drawn to scale. Like reference numbers and designations in the various drawings indicate like elements. For purposes of clarity, not every component may be labeled in every drawing. In the drawings:

FIG. 1 depicts an example system.

FIG. 2 depicts an example vehicle environment.

FIG. 3 depicts an example trip offset user interface.

FIG. 4 depicts an example mobile trip user interface.

FIG. 5 depicts an example mobile profile user interface.

FIG. 6 depicts an example mobile grid user interface.

FIG. 7 depicts an example lifetime offset user interface.

FIG. 8 depicts an example mobile build profile user interface.

FIG. 9 depicts an example mobile lifetime grid user interface.

FIG. 10 depicts an example cross-sectional view of an electric vehicle installed with at least one battery pack.

FIG. 11 is a block diagram illustrating an architecture for a computer system that can be employed to implement elements of the systems and methods described and illustrated herein.

FIG. 12 depicts an example method of mitigating vehicle carbon emission.

### DETAILED DESCRIPTION

Following below are more detailed descriptions of various concepts related to, and implementations of, methods, apparatuses, and systems of mitigating vehicle carbon emission. The various concepts introduced above and discussed in greater detail below may be implemented in any of numerous ways.

The present disclosure is directed to systems and methods of mitigating vehicle carbon emission. The disclosed solutions have a technical improvement of identification of a quantity of carbon emissions associated with a particular vehicle over a particular period of time in view of the particular carbon emissions associated with the vehicle and the electricity supplied to the vehicle. Thus, this technical solution provides a technical improvement beyond the capability of manual analysis of carbon emission. The disclosed solutions have a technical improvement of mitigating a carbon emitted by a vehicle, based on metrics associated with one or more of the vehicle, the components of the vehicle, the operation of the vehicle, the sources of electricity delivered to the vehicle, or any combination thereof. For example, an in-vehicle infotainment system can use data and calculations of real-time tracking and monitoring of vehicle efficiency, data tracking charging history (kWh and time of charging), data of energy consumed by the vehicle and by end use phase, waste energy, and other usage statistics in order to present total emissions and electricity consumption based on individual trips or any duration of use (per week, per month, per year). The user can then be provided a simple transaction process, in vehicle, to offset the trip (of any duration) with purchase of credible carbon offsets (tons co2e) and renewable energy (MWh of renewable energy certificates) that are retired or transferred directly to the user to mitigate the deleterious environmental impacts of the specific reported use phase. For example, a user can finish a road trip and go to their trip information screen in the infotainment system.

For example, the infotainment system can display that a trip contributed to a certain volume of emissions (CO₂e—carbon dioxide equivalent) based on the individual uses and the kWh of fossil-based fuels consumed in charging (using tracked vehicle performance data and tracked charging history data). A user can then be presented with an option to “offset” these emissions and energy use with more environmental friendly alternatives or offset mechanisms that would eliminate the negative impacts of the specified trip. The transaction can occur directly in the vehicle and present the user with a receipt of transaction, outlining where/how their purchase has contributed specifically to avoiding or eliminating emissions through carbon offset purchases and/or renewable energy certificate purchases, outline specific projects that were used to offset the environmental impacts, and other information to help improve/reduce future emissions or energy use (i.e., driving tips or other efficiency tips to improve/reduce impacts of operation). The infotainment system can present the user with an updated emissions and environmental impact report after the purchase of offsets and renewable energy certificates have been made to provide real-time feedback and updated emissions and energy use profile, modified with the mitigation efforts purchased through the transaction platform.

Thus, users can be presented with calculated predictions of real-time impacts in a configuration tool updated in real-time as selections and configuration choices. For example, a configuration choice can indicate emission corresponding to a lifecycle cost of the product. For example, lifecycle cost can correspond to dollars per year of operation, or dollars over lifetime use of the product. For example, a configuration choice can indicate emissions corresponding to a full cradle-to-grave lifecycle emission of the product. For example, lifecycle emission can correspond to tons of carbon dioxide emitted (CO₂e) per year of operation, or tons of carbon dioxide emitted over lifetime use of the product. For example, a configuration choice can indicate emission corresponding to an energy performance and use of the product. Energy performance can correspond to kilowatt-hours (kWh) per year of operation, or kWh over lifetime use of the product. For example, as users make selections, add accessories, or configure the product, just as the first cost updates in the configuration tool, users can also be presented with the impacts of their choices based on lifecycle costs, emissions and energy consumption.

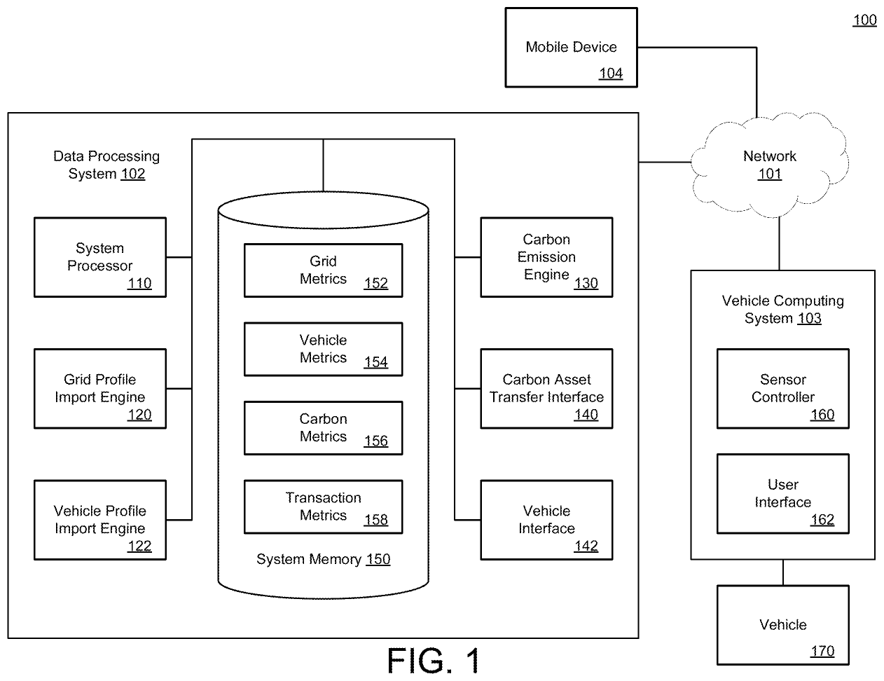

FIG. 1 depicts an example system 100, in accordance with present implementations. As illustrated by way of example in FIG. 1, an example system 100 can include at least a network 101, a data processing system 102, a vehicle computing system 103, and a vehicle 170. The vehicle 170 can include an electric vehicle.

The network 101 can include any type or form of network. The geographical scope of the network 101 can vary widely and the network 101 can include a body area network (BAN), a personal area network (PAN), a local-area network (LAN), e.g. Intranet, a metropolitan area network (MAN), a wide area network (WAN), or the Internet. The topology of the network 101 can be of any form and can include, e.g., any of the following: point-to-point, bus, star, ring, mesh, or tree. The network 101 can include an overlay network which is virtual and sits on top of one or more layers of other networks 101. The network 101 can be of any such network topology as known to those ordinarily skilled in the art capable of supporting the operations described herein. The network 101 can utilize different techniques and layers or stacks of protocols, including, e.g., the Ethernet protocol, the Internet protocol suite (TCP/IP), the ATM (Asynchronous Transfer Mode) technique, the SONET (Synchronous Optical Networking) protocol, or the SD (Synchronous Digital Hierarchy) protocol. The TCP/IP Internet protocol suite can include application layer, transport layer, Internet layer (including, e.g., IPv6), or the link layer. The network 101 can include a type of a broadcast network, a telecommunications network, a data communication network, or a computer network.

The data processing system 102 can include a physical computer system operatively coupled or that can be coupled with one or more components of the system 100, either directly or directly through an intermediate computing device or system. The data processing system 102 can include a virtual computing system, an operating system, and a communication bus to effect communication and processing. The data processing system 102 can include a system processor 110, a grid profile import engine 120, a vehicle profile import engine 122, a carbon emission engine 130, a carbon asset transfer interface 140, a vehicle interface 142, and a system memory 150.

The system processor 110 can execute one or more instructions associated with the system 100. The system processor 110 can include an electronic processor, an integrated circuit, or the like including one or more of digital logic, analog logic, digital sensors, analog sensors, communication buses, volatile memory, nonvolatile memory, and the like. The system processor 110 can include, but is not limited to, at least one microcontroller unit (MCU), microprocessor unit (MPU), central processing unit (CPU), graphics processing unit (GPU), physics processing unit (PPU), embedded controller (EC), or the like. The system processor 110 can include a memory operable to store or storing one or more instructions for operating components of the system processor 110 and operating components operably coupled to the system processor 110. For example, the one or more instructions can include one or more of firmware, software, hardware, operating systems, embedded operating systems. The system processor 110 or the system 100 generally can include one or more communication bus controller to effect communication between the system processor 110 and the other elements of the system 100.

The grid profile import engine 120 can obtain one or more metrics corresponding to a source of electricity provided to the vehicle 170. For example, the grid profile import engine 120 can obtain metrics corresponding to carbon emissions generated by a source of power linked with the vehicle 170 via a particular charging station. The grid profile import engine 120 can identify a particular charging station or stations from which the vehicle 170 has obtained or is obtaining power. For example, the grid profile import engine 120 can identify a location associated with a particular charger, and can determine a number of sources of power and the percentage of each source of power provided to the portion of the electrical gird coupled with the particular charger. The grid profile import engine 120 can communicate with a grid system external to the system 100 to obtain metrics corresponding to particular sources of power. For example, the grid profile import engine 120 can include a communication interface configured to transmit a location corresponding to the vehicle in a format compatible with the grid system. For example, the grid profile import engine 120 can communicate with the grid system by an application programming interface (“API”) configured to transmit location metrics corresponding to the vehicle 170, a charging station coupled with or can couple with the vehicle 170, or any combination thereof.

The vehicle profile import engine 122 can obtain one or more metrics corresponding to the vehicle 170, components of the vehicle 170, or any combination thereof. For example, the vehicle profile import engine 122 can obtain metrics corresponding to carbon emissions generated by use of the vehicle 170 or a particular component of the vehicle 170. For example, the vehicle profile import engine 122 can obtain metrics corresponding to carbon emissions generated by construction of the vehicle 170 or a particular component of the vehicle 170. The vehicle profile import engine 122 can identify a particular battery, tire, electrical system, or any combination thereof, for example, and determine an electrical consumption by one or more of those systems based on usage metrics obtained from one or more sensors of the vehicle 170. For example, sensors of the vehicle 170 can include electrical sensors configured to detect electricity provided to or expended by particular components of the vehicle 170.

For example, the vehicle profile import engine 122 can include a communication interface configured to identify characteristics of the vehicle 170 in a format compatible with the vehicle 170. For example, the vehicle profile import engine 122 can communicate with the vehicle 170 by an application programming interface (“API”) configured to identify characteristics of the vehicle 170 corresponding to electricity consumption by the particular vehicle 170. For example, the particular vehicle 170 can correspond to an individual vehicle, an individual vehicle customized according to an individual modification, a model of the vehicle 170, or a customization of a model of the vehicle 170 according to a customization profile of a manufacturer.

The carbon emission engine 130 can identify carbon emissions associated with generation of the electricity provided to the vehicle 170 by the particular charger. The carbon emission engine 130 can determine an amount of carbon emission (e.g., CO₂e) based on the amount of electricity delivered to the vehicle 170 by the particular charger, and the carbon emission associated with the source of power to the particular charger. For example, a source of power can correspond to a mix of power plants for a particular grid region. For example, the carbon emission engine 130 can obtain electricity metrics corresponding to one or more of electricity applied to a vehicle, electricity supplied by a charging device, electricity loss through transfer via a grid from a source of power to the charging device, electricity loss through transfer via a charging station to the vehicle 170, or any combination thereof. For example, electricity metrics can include the carbon emission engine 130 which can convert an electricity metric into a emission metric based on various source metrics. For example, an emission metric can correspond to an amount of carbon emitted in response to generation or transfer of electricity from a particular source of power via a particular grid to a particular vehicle. For example, the system 100 can obtain, from the vehicle 170 and via a first communication interface compatible with the source of power, an identifier of the source of power. For example, the system can include the emission metric corresponding to an estimated amount of carbon emitted by the vehicle over a trip of the vehicle. For example, the system can include the emission metric corresponding to an estimated amount of carbon emitted by the vehicle over a lifetime of the vehicle. The system 100 can obtain, based on the identifier and via a second communication interface compatible with a third system, a second emission metric corresponding to the source of power. The system 100 can determine, based on the second emission metric, the emission metric. Thus, this technical solution can provide at least the technical improvement of determination of carbon emissions corresponding to particular vehicle electricity consumption in real-time or contemporaneous with completion of travel, at a speed and accuracy beyond the capability of manual processes to determine.

The carbon asset transfer interface 140 can execute one or more instructions to mitigate the physical impact of carbon emissions corresponding to the emission metric. For example, the carbon asset transfer interface 140 can execute instructions to reduce environmental carbon, in the form of carbon dioxide in the planetary atmosphere, via execution of instructions to transfer assets corresponding to the emission metric. For example, the carbon asset transfer interface 140 can interface with an external system configured to transfer, activate, purchase, or create carbon assets. For example, the carbon assets can correspond to carbon credits. For example, the system can correspond to an asset having an aggregate value to offset the amount of carbon emitted by the vehicle. The carbon asset transfer interface 140 can cause a reduction of an amount of carbon dioxide in the planetary atmosphere by an amount corresponding to the emission metric. Thus, the carbon asset transfer interface 140 can provide at least a technical improvement of a reduction of an amount of carbon dioxide in the planetary atmosphere, at a speed and accuracy corresponding to a vehicle impact, beyond the capability of manual processes to effect.

The vehicle interface 142 can include a communication interface configured to request and obtain sensor data from the vehicle 170 in a format compatible with the vehicle 170. For example, the vehicle interface 142 can communicate with the vehicle 170 by an application programming interface (“API”) configured to transmit sensor data corresponding to a particular battery, tire, electrical system, or any combination thereof. Thus, the vehicle interface 142 can interface with the vehicle 170 to identify characteristics of the vehicle 170 corresponding to contemporaneous or real-time electricity consumption by the particular vehicle 170.

The system memory 150 can store data associated with the system 100. The system memory 150 can include one or more hardware memory devices to store binary data, digital data, or the like. The system memory 150 can include one or more electrical components, electronic components, programmable electronic components, reprogrammable electronic components, integrated circuits, semiconductor devices, flip flops, arithmetic units, or the like. The system memory 150 can include at least one of a non-volatile memory device, a solid-state memory device, a flash memory device, or a NAND memory device. The system memory 150 can include one or more addressable memory regions disposed on one or more physical memory arrays. A physical memory array can include a NAND gate array disposed on, for example, at least one of a particular semiconductor device, integrated circuit device, and printed circuit board device. The system memory 150 can include storage portions for grid metrics 152, vehicle metrics 154, carbon metrics 156, and transaction metrics 156.

The grid metrics 152 can correspond to identifications of sources of power, identifications of grids associated with particular sources of power, percentages of sources of power associated with particular grid or grid portions, amount or rate of carbon emitted by particular sources of power, amount or rate of power generated by particular sources of power, locations associated with particular grids or grid portions, locations associated with particular charging stations, correspondences between particular charging stations, location, grids or gird portions, and sources of power. The vehicle metrics 154 can correspond to identification of the vehicle 170 or any component thereof. For example, the vehicle metrics 154 can correspond to a model of the vehicle 170, an identification of the vehicle 170, an identification of components of the vehicle. The vehicle metrics 154 can include metrics corresponding to one or more vehicles, and are not limited to the vehicle 170.

The carbon metrics 156 can correspond to emission metrics corresponding to the vehicle 170 or any component thereof, identifications of particular emissions metrics corresponding to the vehicle 170 or the components thereof. For example, the carbon metrics 156 can include an amount of carbon associated with build or manufacture of the vehicle 170 or any component thereof. For example, the carbon metrics 156 can include an amount of carbon associated with use or operation of the vehicle 170 or any component thereof over a particular period of time. For example, the carbon metrics 156 can include an amount of carbon associated with use or operation of the vehicle 170 or any component thereof, a particular rate of consumption of electricity, including but not limited to operation at particular speeds or according to particular driving patterns. For example, the system 100 can determine, based on a source metric identifying the source of power, the emission metric. For example, the system 100 can determine, based on a second emission metric corresponding to a component of the vehicle 170, the emission metric. The transaction metrics 156 can identify carbon assets, carbon asset systems, keys or credential corresponding to interfaces compatible with particular, carbon asset systems, or any combination thereof. For example, the transaction metrics 156 can identify particular carbon asset systems configured to transfer, purchase or activate carbon assets to mitigate carbon emission corresponding to an emission metric.

The vehicle computing system 103 can communicate with the data processing system 102 by the network 101, and can communicate with the vehicle 170 by one or more communication protocols therebetween. The vehicle computing system 103 can include a sensor controller 160, and a user interface 162.

The sensor controller 160 can include a communication interface configured to transmit sensor data from the vehicle 170 in a format compatible with the vehicle 170. For example, the vehicle interface 142 can communicate with the vehicle 170 by an API corresponding to the vehicle interface 142 configured to transmit sensor data corresponding to a particular battery, tire, electrical system, or any combination thereof. Thus, the sensor controller 160 can interface with the vehicle interface 142 to identify characteristics of the vehicle 170 corresponding to contemporaneous or real-time electricity consumption by the particular vehicle 170. The sensor controller 160 can include a communication interface configured to identify characteristics of the vehicle 170 in a format compatible with the vehicle 170. For example, the sensor controller 160 can communicate with the vehicle 170 by an API corresponding to the vehicle profile import engine 122 configured to identify characteristics of the vehicle 170 corresponding to electricity consumption by the particular vehicle 170. Thus, the sensor controller 160 can provide sensor-level or component-level metrics from the vehicle 170 to the data processing system, by one or more communication interfaces in a format compatible with transceivers of the sensors or components of the vehicle 170.

For example, the system 100 can obtain, from the vehicle 170 and via a communication interface compatible with the vehicle 170, a vehicle metric corresponding to a component of the vehicle 170. The system 100 can determine, based on the vehicle 170 metric, a second emission metric corresponding to the component of the vehicle 170. The system 100 can determine, based on the second emission metric, the emission metric. For example, the system 100 can obtain, from the vehicle 170 and via a communication interface compatible with the vehicle 170, a vehicle metric corresponding to a component of the vehicle 170. The system 100 can determine, based on the vehicle 170 metric, a second emission metric corresponding to the component of the vehicle 170. The system 100 can determine, based on the second emission metric, the emission metric.

The user interface 162 can include a user interface presentable on a display device operatively coupled with or integrated with the vehicle 170. For example, the display can be integrated with a dashboard of the vehicle 170. The display can display at least one or more user interface presentations and control affordances, and can include an electronic display. An electronic display can include, for example, a liquid crystal display (LCD), a light-emitting diode (LED) display, an organic light-emitting diode (OLED) display, or the like. The display device can be housed at least partially within the vehicle 170.

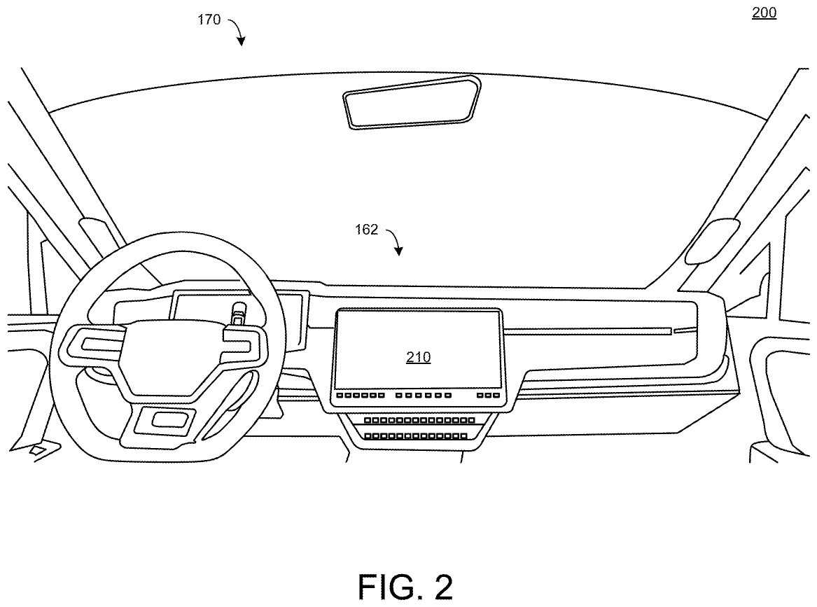

FIG. 2 depicts an example vehicle environment 200, in accordance with present implementations. As illustrated by way of example in FIG. 2, an example vehicle environment 200 can include at least a vehicle display device 210. The vehicle display device 210 can correspond at least partially in one or more of structure and operation to a display of the user interface 162. For example, the vehicle display device 210 can include a touchscreen display configured to present at least a portion of any user interface depicted or discussed herein, but is not limited thereto.

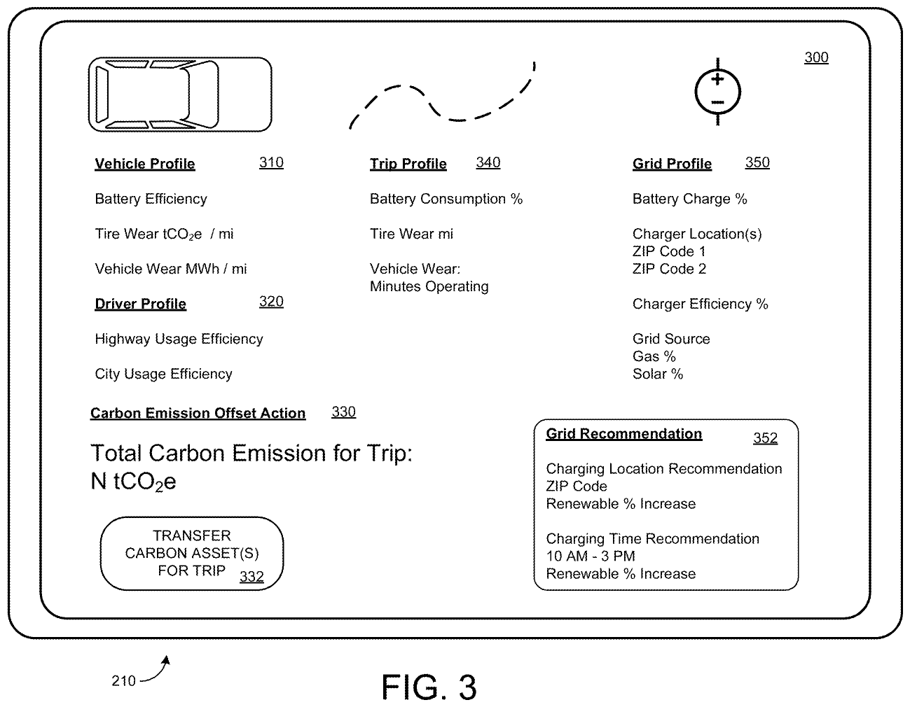

FIG. 3 depicts an example trip offset user interface 300, in accordance with present implementations. As illustrated by way of example in FIG. 3, an example trip offset user interface 300 can include at least a vehicle profile presentation 310, a driver profile presentation 320, a carbon offset presentation 330, a trip profile presentation 340, and a grid profile presentation 350. For example, the example trip offset user interface 300 can present one or more indications corresponding to carbon emissions generated by operation of a particular vehicle in a particular time period and based on power delivered to the particular vehicle from particular sources of power. Thus, the example trip offset user interface 300 can present indications of carbon emissions customized to the operation of the vehicle 170. For example, a particular vehicle can correspond to the vehicle 170.

The vehicle profile presentation 310 can present one or more indications corresponding to one or more metrics dependent on a particular vehicle. For example, metrics dependent on a particular vehicle can include electricity metrics or emission metrics based on the vehicle. For example, the vehicle profile presentation 310 can present a battery efficiency presentation indicating an amount of power corresponding to a distance of travel or time of operation. For example, the vehicle profile presentation 310 can present a tire wear presentation indicating an amount of carbon emitted by tire degradation with respect to distance of travel or time of operation. For example, the vehicle profile presentation 310 can present a vehicle wear presentation indicating an amount of carbon emitted by vehicle degradation with respect to distance of travel or time of operation. For example, the vehicle profile presentation 310 can present one or more indications via the carbon emission engine 130 and the vehicle metrics 154.

The driver profile presentation 320 can present one or more indications corresponding to one or more metrics dependent on a particular user of a particular vehicle. For example, metrics dependent on a particular user of a particular vehicle can include electricity metrics or emission metrics based on historical driving data of the user. For example, the driver profile presentation 320 can present a highway usage efficiency presentation indicating an amount of power consumed on average by a user of the vehicle while operating above a highway threshold speed or along particular roadways designated as highways. For example, the driver profile presentation 320 can present a city usage efficiency presentation indicating an amount of power consumed on average by a user of the vehicle while operating below a highway speed, above a minimum threshold speed, or along particular roadways designated as surface streets. For example, the driver profile presentation 320 can present one or more indications via the carbon emission engine 130 and the vehicle metrics 154.

The carbon offset presentation 330 can present one or more indications corresponding to one or more metrics dependent on operation of a particular vehicle over a particular time period or between one or more particular locations. For example, metrics dependent on operation of a particular vehicle over a particular time period or between one or more particular locations can include an aggregate of emission metrics based on a path of travel from a first physical location to one or more second physical location, and one or more times of travel corresponding to the path or any portion thereof. For example, the carbon offset presentation 330 can present a total carbon emission presentation indicating an amount of carbon emitted in response to operation of the vehicle over the course of a particular trip or portion thereof. For example, the carbon offset presentation 330 can present one or more indications of an amount in carbon of tons of CO₂ emitted in response to operation of the vehicle. For example, the carbon offset presentation 330 can present one or more indications via the carbon emission engine 130, the grid metrics 152, the vehicle metrics 154, and the carbon metrics 156. The carbon offset presentation 330 can include a carbon offset control affordance 332.

The carbon offset control affordance 332 can include a portion of the trip offset user interface 300 configured to receive input via the user interface 162. For example, the carbon offset control affordance 332 corresponds to a button presentation configured to receive user input to activate or actuate the carbon offset control affordance 332. In response to the user input, the carbon offset control affordance 332 can cause the carbon asset transfer interface 140 to obtain an offset metric corresponding to an amount of carbon indicated by the carbon offset presentation 330. In response to the user input, the carbon offset control affordance 332 can cause the carbon asset transfer interface 140 transfer or purchase a carbon asset corresponding to an offset metric corresponding to an amount of carbon indicated by the carbon offset presentation 330. The carbon offset control affordance 332 can cause the carbon asset transfer interface 140 transfer or purchase a carbon asset based on one or more of the transaction metrics 158. Thus, the carbon offset control affordance 332 can achieve a technical solution to mitigate carbon dioxide in the planetary atmosphere by an amount corresponding to the operation of a particular vehicle over a particular time period or between one or more particular locations.

The trip profile presentation 340 can present one or more indications corresponding to one or more metrics dependent on operation of a particular vehicle over a particular time period or between one or more particular locations. For example, metrics dependent on operation of a particular vehicle over a particular time period or between one or more particular locations can include electricity metrics or emission metrics based on a path of travel from a first physical location to one or more second physical locations, and one or more times of travel corresponding to the path or any portion thereof. For example, the trip profile presentation 340 can present a battery consumption presentation indicating an amount of charge depleted by the battery of the vehicle over the course of a particular trip or portion thereof. For example, the vehicle profile presentation 310 can present one or more indications via the carbon emission engine 130 and the vehicle metrics 154.

The grid profile presentation 350 can present one or more indications corresponding to one or more metrics dependent on electricity delivered to a particular vehicle over a particular time period or between one or more particular locations. For example, metrics dependent on electricity delivered to a particular vehicle over a particular time period or between one or more particular locations can include electricity metrics or emission metrics based on charging of a battery of a vehicle before, during, or after a path of travel from a first physical location to one or more second physical locations, and one or more times of travel corresponding to the path or any portion thereof. For example, the grid profile presentation 350 can present a battery charge presentation indicating an amount of charge provided to the battery of the vehicle over the course of a particular trip or portion thereof. For example, the grid profile presentation 350 can present a charger location presentation indicating a location corresponding to charging stations from which charge is provided to the battery of the vehicle in connection with or over the course of a particular trip or portion thereof. For example, the grid profile presentation 350 can present a charger efficiency presentation indicating one or more efficiency metrics each corresponding to various charging stations from which charge is provided to the battery of the vehicle in connection with or over the course of a particular trip or portion thereof. For example, the grid profile presentation 350 can present a grid carbon composition presentation indicating carbon emission types corresponding to one or more sources of power linked with charging stations from which charge is provided to the battery of the vehicle in connection with or over the course of a particular trip or portion thereof. The grid profile presentation 350 can present one or more indications via the grid profile import engine 120 and the grid metrics 152. The grid recommendation presentation 352 can include a grid recommendation presentation 352.

The grid recommendation presentation 352 can present one or more indications corresponding to modification to charging. For example, the grid recommendation presentation 352 can include a location recommendation presentation including a charging station corresponding to a recommended emission metric lower than an emission metric corresponding to a trip. For example, the grid recommendation presentation 352 can include a timing recommendation presentation including a time period for a charging station. The timing recommendation presentation can correspond to a recommended emission metric lower than an emission metric corresponding to a trip. The grid recommendation presentation 352 can present one or more indications via the carbon emission engine 130, and the grid metrics 152, the vehicle metrics 154 and the carbon metrics 156.

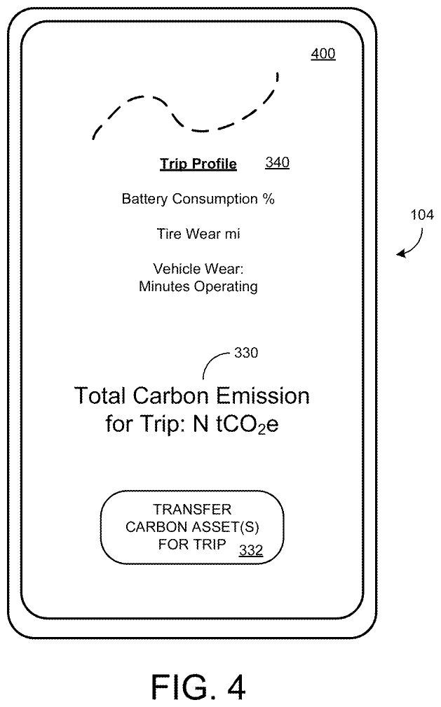

FIG. 4 depicts an example mobile trip user interface 400, in accordance with present implementations. As illustrated by way of example in FIG. 4, an example mobile trip user interface 400 can include a portion of the trip offset user interface 300. The mobile trip user interface 400 can be presented by the mobile device 104. For example, the mobile device 104 can present the trip profile presentation 340, and the carbon offset presentation 330 including the carbon offset control affordance 332.

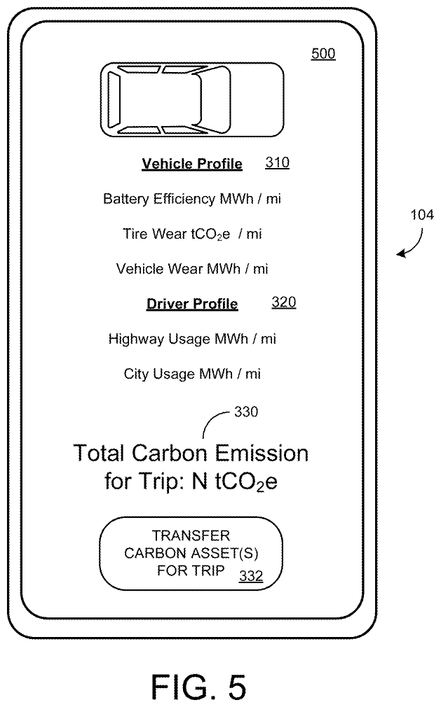

FIG. 5 depicts an example mobile profile user interface 500, in accordance with present implementations. As illustrated by way of example in FIG. 5, an example mobile profile user interface 500 can include a portion of the trip offset user interface 300. The mobile profile user interface 500 can be presented by the mobile device 104. For example, the mobile device 104 can present the vehicle profile presentation 310, the driver profile presentation 320, and the carbon offset presentation 330 including the carbon offset control affordance 332.

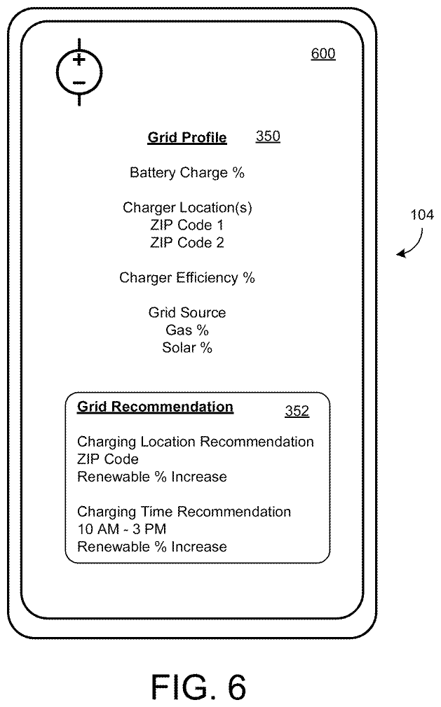

FIG. 6 depicts an example mobile grid user interface 600, in accordance with present implementations. As illustrated by way of example in FIG. 6, an example mobile grid user interface 600 can include a portion of the trip offset user interface 300. The mobile grid user interface 600 can be presented by the mobile device 104. For example, the mobile device 104 can present the grid profile presentation 350 including the grid recommendation presentation 352.

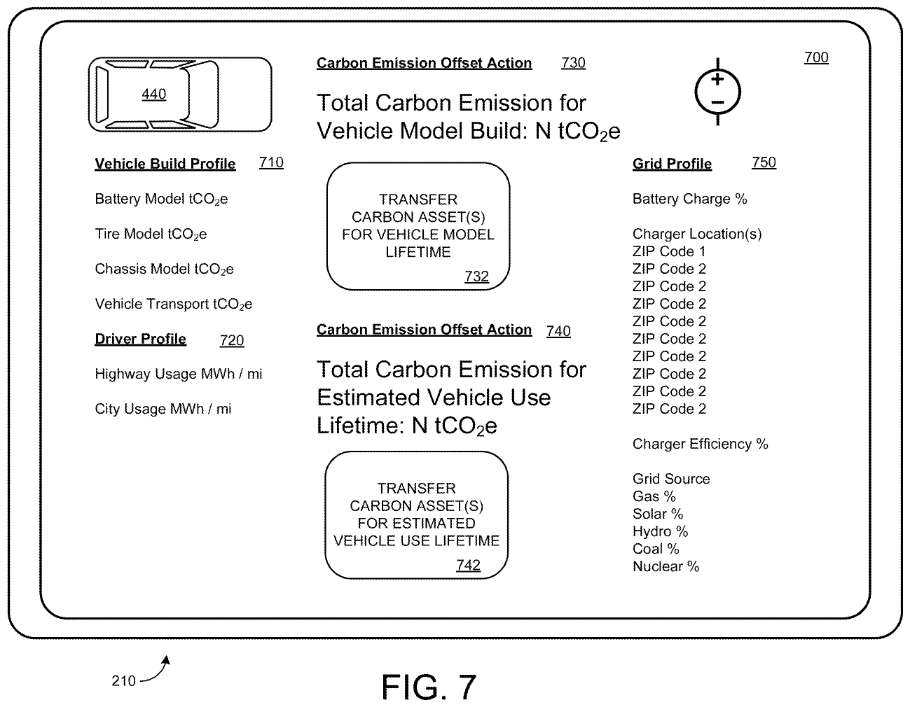

FIG. 7 depicts an example lifetime offset user interface 700, in accordance with present implementations. As illustrated by way of example in FIG. 7, an example lifetime offset user interface 700 can include at least a vehicle profile presentation 710, a driver profile presentation 720, a build offset presentation 730, a use offset presentation 740, and a grid profile presentation 750.

The vehicle profile presentation 710 can present one or more indications corresponding to one or more metrics dependent on a particular vehicle. For example, metrics dependent on a particular vehicle can include electricity metrics or emission metrics based on manufacture of the vehicle or components of the vehicle. For example, the vehicle profile presentation 710 can present one or more of a battery model emission metric, a tire model emission metric, a chassis model emission metric, and a vehicle transport emission metric. The battery model emission metric, the tire model emission metric, the chassis model emission metric, and the vehicle transport emission metric can correspond to respectively amounts of carbon emitted to manufacture or build the battery, tires, chassis of the vehicle or to transport the vehicle to its final destination for sale. For example, the vehicle profile presentation 710 can present one or more indications via the carbon emission engine 130 and the vehicle metrics 154. The driver profile presentation 720 can correspond at least partially in one or more of structure and operation to the driver profile presentation 320. For example, the driver profile presentation 720 can present metrics corresponding to an estimate of carbon emissions over the lifetime of a particular vehicle according to a vehicle build profile of the vehicle profile presentation 710.

The build offset presentation 730 can present one or more indications corresponding to one or more metrics dependent on manufacture of a particular vehicle or one or more components thereof. For example, metrics dependent on manufacture of a particular vehicle or one or more components thereof can include emission metrics based on carbon emitted to manufacture various components of the vehicle, to assemble the vehicle, and to transport the vehicle. For example, the build offset presentation 730 can present a total carbon emission presentation indicating an amount of carbon emitted in response to manufacture of a particular vehicle including components specific to that vehicle. For example, the build offset presentation 730 can present one or more indications of an amount in carbon of tons of CO₂ emitted in response to manufacture of the vehicle. For example, the build offset presentation 730 can present one or more indications via the carbon emission engine 130, the grid metrics 152, the vehicle metrics 154, and the carbon metrics 156. The build offset presentation 730 can include a build offset control affordance 732.

The build offset control affordance 732 can correspond at least partially in one or more of structure and operation to the carbon offset control affordance 332. In response to user input, the build offset control affordance 732 can cause the carbon asset transfer interface 140 obtain a build offset metric corresponding to an amount of carbon indicated by the build offset presentation 730. In response to the user input, the build offset control affordance 732 can cause the carbon asset transfer interface 140 transfer or purchase a carbon asset corresponding to the build offset metric corresponding to an amount of carbon indicated by the build offset presentation 730. The build offset control affordance 732 can cause the carbon asset transfer interface 140 transfer or purchase a carbon asset based on one or more of the transaction metrics 158. Thus, the build offset control affordance 732 can achieve a technical solution to mitigate carbon dioxide in the planetary atmosphere by an amount corresponding to the manufacture of a particular vehicle.

The use offset presentation 740 can present one or more indications corresponding to one or more metrics dependent on operation of a particular vehicle over a lifetime time period or between one or more particular locations. For example, metrics dependent on operation of a particular vehicle over a lifetime time period or between one or more particular locations can include an aggregate of emission metrics based on aggregate mileage or operation of a vehicle over a predetermined number of months or years, or according to a projected useful life of the vehicle. For example, the carbon offset presentation 330 can present a total carbon emission presentation indicating an amount of carbon emitted in response to operation of the vehicle over the course of a useful life of the vehicle. For example, the carbon offset presentation 330 can present one or more indications of an amount in carbon of tons of CO₂ estimated to be emitted in response to operation of the vehicle over the lifetime time period. For example, the use offset presentation 740 can present one or more indications via the carbon emission engine 130, the grid metrics 152, the vehicle metrics 154, and the carbon metrics 156. The use offset presentation 740 can include a use offset control affordance 742.

The use offset control affordance 742 can correspond at least partially in one or more of structure and operation to the carbon offset control affordance 332. In response to user input, the use offset control affordance 742 can cause the carbon asset transfer interface 140 obtain a use offset metric corresponding to an amount of carbon indicated by the use offset presentation 740. In response to the user input, the use offset control affordance 742 can cause the carbon asset transfer interface 140 transfer or purchase a carbon asset corresponding to the use offset metric corresponding to an amount of carbon indicated by the use offset presentation 740. The use offset control affordance 742 can cause the carbon asset transfer interface 140 transfer or purchase a carbon asset based on one or more of the transaction metrics 158. Thus, the use offset control affordance 742 can achieve a technical solution to mitigate carbon dioxide in the planetary atmosphere by an amount corresponding to lifetime use of a particular vehicle.

The grid profile presentation 750 can correspond at least partially in one or more of structure and operation to the grid profile presentation 350. The grid profile presentation 750 can present one or more indications corresponding to one or more metrics dependent on electricity delivered to a particular vehicle over a lifetime time period. For example, metrics dependent on electricity delivered to a particular vehicle over a lifetime time period can include electricity metrics or emission metrics based on projected charging of a battery of a vehicle corresponding to a particular portion of a grid or charging stations of a grid within a driving range of an expected origin location of a user the vehicle. For example, the grid profile presentation 350 can present a grid carbon composition presentation indicating carbon emission types corresponding to one or more sources of power linked with charging stations from which charge can be provided to the battery of the vehicle within the range during the lifetime time period. The grid profile presentation 350 can present one or more indications via the grid profile import engine 120 and the grid metrics 152.

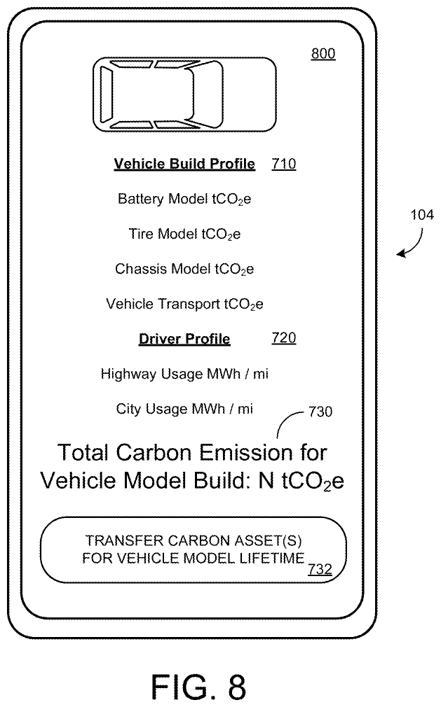

FIG. 8 depicts an example mobile build profile user interface 800, in accordance with present implementations. As illustrated by way of example in FIG. 8, an example mobile build profile user interface 800 can include a portion of the lifetime offset user interface 700. The mobile build profile user interface 800 can be presented by the mobile device 104. For example, the mobile device 104 can present the vehicle build profile presentation 710, the driver profile presentation 720, and the build offset presentation 730 including the build offset control affordance 732.

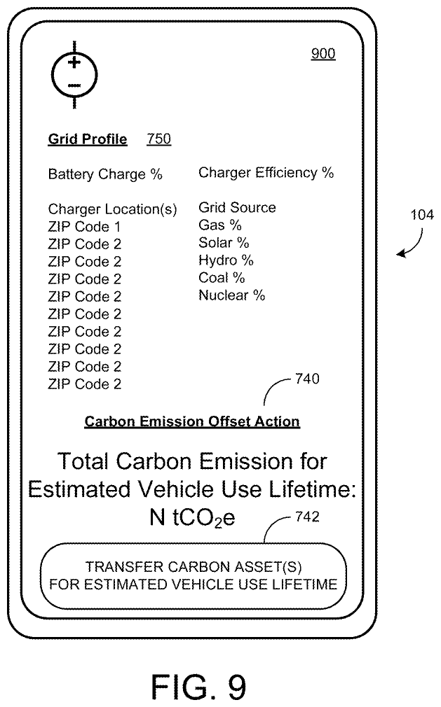

FIG. 9 depicts an example mobile lifetime grid user interface 900, in accordance with present implementations. As illustrated by way of example in FIG. 9, an example mobile lifetime grid user interface 900 can include a portion of the lifetime offset user interface 700. The mobile lifetime grid user interface 900 can be presented by the mobile device 104. For example, the mobile device 104 can present the grid profile presentation 750 and the use offset presentation 740 including the use offset control affordance 742.

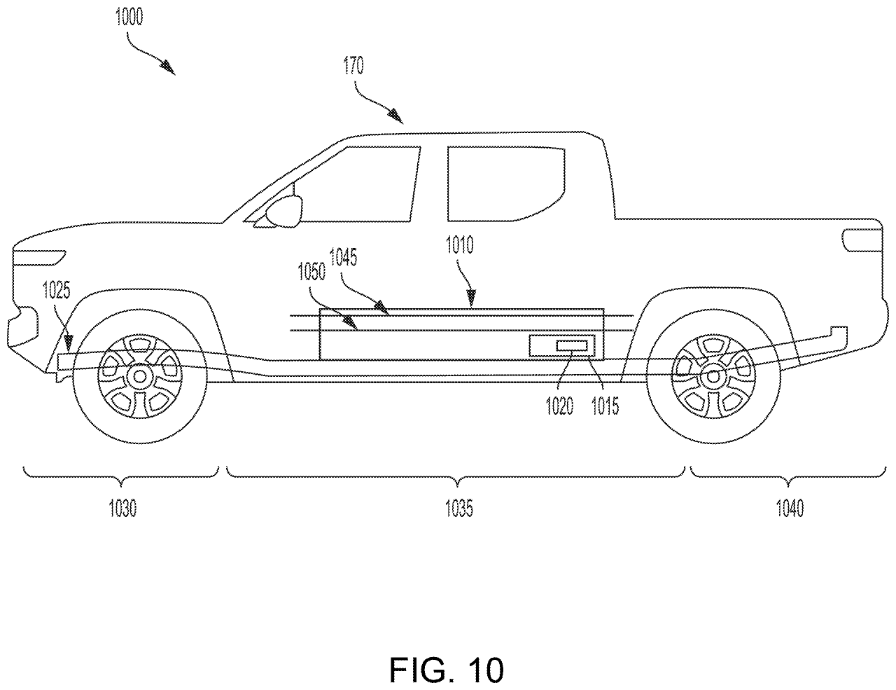

FIG. 10 depicts an example cross-sectional view 1000 of an electric vehicle 170 installed with at least one battery pack 1010. Electric vehicles 170 can include electric trucks, electric sport utility vehicles (SUVs), electric delivery vans, electric automobiles, electric cars, electric motorcycles, electric scooters, electric passenger vehicles, electric passenger or commercial trucks, hybrid vehicles, or other vehicles such as sea or air transport vehicles, planes, helicopters, submarines, boats, or drones, among other possibilities. The battery pack 1010 can also be used as an energy storage system to power a building, such as a residential home or commercial building. Electric vehicles 170 can be fully electric or partially electric (e.g., plug-in hybrid) and further, electric vehicles 170 can be fully autonomous, partially autonomous, or unmanned. Electric vehicles 170 can also be human operated or non-autonomous. Electric vehicles 170 such as electric trucks or automobiles can include on-board battery packs 1010, batteries 1015 or battery modules 1015, or battery cells 1020 to power the electric vehicles. The electric vehicle 170 can include a chassis 1025 (e.g., a frame, internal frame, or support structure). The chassis 1025 can support various components of the electric vehicle 170. The chassis 1025 can span a front portion 1030 (e.g., a hood or bonnet portion), a body portion 1035, and a rear portion 1040 (e.g., a trunk, payload, or boot portion) of the electric vehicle 170. The battery pack 1010 can be installed or placed within the electric vehicle 170. For example, the battery pack 1010 can be installed on the chassis 1025 of the electric vehicle 170 within one or more of the front portion 1030, the body portion 1035, or the rear portion 1040. The battery pack 1010 can include or connect with at least one busbar, e.g., a current collector element. For example, the first busbar 1045 and the second busbar 150 can include electrically conductive material to connect or otherwise electrically couple the battery 1015, the battery modules 1015, or the battery cells 1020 with other electrical components of the electric vehicle 170 to provide electrical power to various systems or components of the electric vehicle 170.

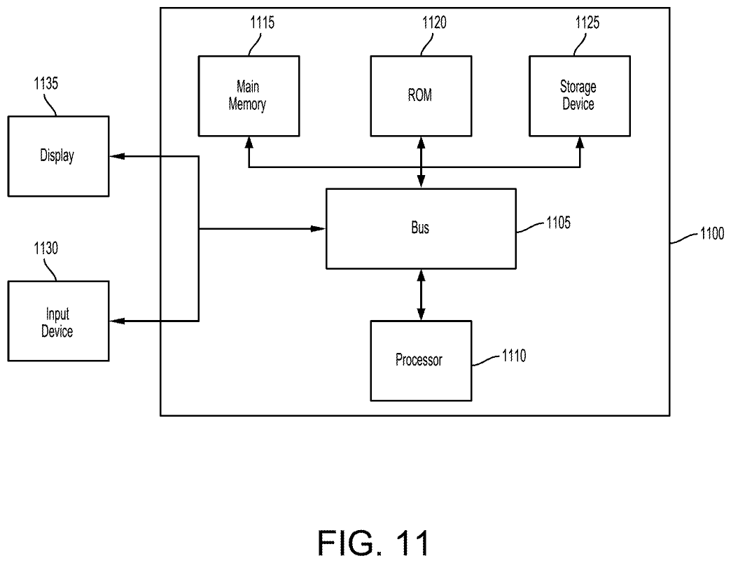

FIG. 11 depicts an example computer system, in accordance with present implementations. The computer system 1100 or computing device can include or be used to implement a data processing system or its components. The computing system 1100 includes at least one bus 1105 or other communication component for communicating information and at least one processor 1110 or processing circuit coupled to the bus 1105 for processing information. The computing system 1100 can also include one or more processors 1110 or processing circuits coupled to the bus for processing information. The computing system 1100 also includes at least one main memory 1115, such as a random access memory (RAM) or other dynamic storage device, coupled to the bus 1105 for storing information, and instructions to be executed by the processor 1110. The main memory 1115 can be used for storing information during execution of instructions by the processor 1110. The computing system 1100 may further include at least one read only memory (ROM) 1120 or other static storage device coupled to the bus 1105 for storing static information and instructions for the processor 1110. A storage device 1125, such as a solid state device, magnetic disk or optical disk, can be coupled to the bus 1105 to persistently store information and instructions.

The computing system 1100 may be coupled via the bus 1105 to a display 1135, such as a liquid crystal display, or active matrix display, for displaying information to a user such as a driver of the electric vehicle 105 or other end user. An input device 1130, such as a keyboard or voice interface may be coupled to the bus 1105 for communicating information and commands to the processor 1110. The input device 1130 can include a touch screen display 1135. The input device 1130 can also include a cursor control, such as a mouse, a trackball, or cursor direction keys, for communicating direction information and command selections to the processor 1110 and for controlling cursor movement on the display 1135.

The processes, systems and methods described herein can be implemented by the computing system 1100 in response to the processor 1110 executing an arrangement of instructions contained in main memory 1115. Such instructions can be read into main memory 1115 from another computer-readable medium, such as the storage device 1125. Execution of the arrangement of instructions contained in main memory 1115 causes the computing system 1100 to perform the illustrative processes described herein. One or more processors in a multi-processing arrangement may also be employed to execute the instructions contained in main memory 1115. Hard-wired circuitry can be used in place of or in combination with software instructions together with the systems and methods described herein. Systems and methods described herein are not limited to any specific combination of hardware circuitry and software.

Although an example computing system has been described in FIG. 3, the subject matter including the operations described in this specification can be implemented in other types of digital electronic circuitry, or in computer software, firmware, or hardware, including the structures disclosed in this specification and their structural equivalents, or in combinations of one or more of them. For example, a computer readable medium can include one or more instructions executable by a processor to obtain, from the vehicle and via a communication interface compatible with the vehicle, a vehicle metric corresponding to a component of the vehicle. The processor can determine, based on the vehicle metric, a second emission metric corresponding to the component of the vehicle. The processor can determine, based on the second emission metric, the emission metric.

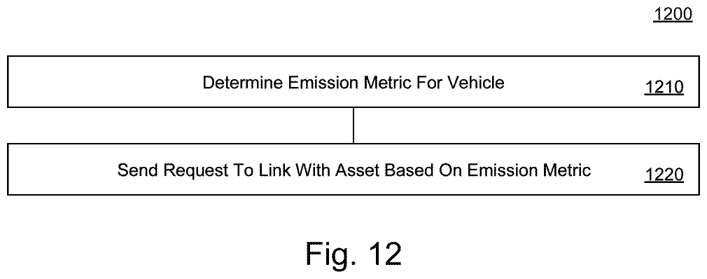

FIG. 12 depicts an example method 1200 of mitigating vehicle carbon emission, in accordance with present implementations. At least the system 100 or any component thereof can perform method 1200.

The method 1200 can determine an emission metric for the vehicle. (Act 1210). For example, the carbon emission engine 130 can determine the emission metric as discussed herein. For example, the emission metric can correspond to an estimated amount of carbon emitted by the vehicle over a trip of the vehicle. For example, the emission metric can correspond to an estimated amount of carbon emitted by the vehicle over a lifetime of the vehicle. For example, the method can include determining, based on a source metric identifying the source of power, the emission metric. For example, the method can include determining, based on a second emission metric corresponding to a component of the vehicle, the emission metric.

For example, the method can include obtaining, from the vehicle and via a communication interface compatible with the vehicle, a vehicle metric corresponding to a component of the vehicle. The method can include determining, based on the vehicle metric, a second emission metric corresponding to the component of the vehicle. The method can include determining, based on the second emission metric, the emission metric. For example, the method can include obtaining, from the vehicle and via a communication interface compatible with the vehicle, a vehicle metric corresponding to a component of the vehicle. The method can include determining, based on the vehicle metric, a second emission metric corresponding to the component of the vehicle. The method can include determining, based on the second emission metric, the emission metric. For example, the method can include obtaining, from the vehicle and via a first communication interface compatible with the source of power, an identifier of the source of power. The method can include obtaining, based on the identifier and via a second communication interface compatible with a third system, a second emission metric corresponding to the source of power. The method can include determining, based on the second emission metric, the emission metric.

The method 1200 can send a request to link with an asset based on the emission metric. (Act 1220). For example, the asset can have an aggregate value to offset the amount of carbon emitted by the vehicle. For example, the carbon asset transfer interface 142 can send a request according to operation or structure of the carbon asset transfer interface 142 as discussed herein.

Some of the description herein emphasizes the structural independence of the aspects of the system components or groupings of operations and responsibilities of these system components. Other groupings that execute similar overall operations are within the scope of the present application. Modules can be implemented in hardware or as computer instructions on a non-transient computer readable storage medium, and modules can be distributed across various hardware or computer based components.

The systems described above can provide multiple ones of any or each of those components and these components can be provided on either a standalone system or on multiple instantiations in a distributed system. In addition, the systems and methods described above can be provided as one or more computer-readable programs or executable instructions embodied on or in one or more articles of manufacture. The article of manufacture can be cloud storage, a hard disk, a CD-ROM, a flash memory card, a PROM, a RAM, a ROM, or a magnetic tape. In general, the computer-readable programs can be implemented in any programming language, such as LISP, PERL, C, C++, C#, PROLOG, or in any byte code language such as JAVA. The software programs or executable instructions can be stored on or in one or more articles of manufacture as object code.

Example and non-limiting module implementation elements include sensors providing any value determined herein, sensors providing any value that is a precursor to a value determined herein, datalink or network hardware including communication chips, oscillating crystals, communication links, cables, twisted pair wiring, coaxial wiring, shielded wiring, transmitters, receivers, or transceivers, logic circuits, hard-wired logic circuits, reconfigurable logic circuits in a particular non-transient state configured according to the module specification, any actuator including at least an electrical, hydraulic, or pneumatic actuator, a solenoid, an op-amp, analog control elements (springs, filters, integrators, adders, dividers, gain elements), or digital control elements.

The subject matter and the operations described in this specification can be implemented in digital electronic circuitry, or in computer software, firmware, or hardware, including the structures disclosed in this specification and their structural equivalents, or in combinations of one or more of them. The subject matter described in this specification can be implemented as one or more computer programs, e.g., one or more circuits of computer program instructions, encoded on one or more computer storage media for execution by, or to control the operation of, data processing apparatuses. Alternatively or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. While a computer storage medium is not a propagated signal, a computer storage medium can be a source or destination of computer program instructions encoded in an artificially generated propagated signal. The computer storage medium can also be, or be included in, one or more separate components or media (e.g., multiple CDs, disks, or other storage devices include cloud storage). The operations described in this specification can be implemented as operations performed by a data processing apparatus on data stored on one or more computer-readable storage devices or received from other sources.

The terms “computing device”, “component” or “data processing apparatus” or the like encompass various apparatuses, devices, and machines for processing data, including by way of example a programmable processor, a computer, a system on a chip, or multiple ones, or combinations of the foregoing. The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, app, script, or code) can be written in any form of programming language, including compiled or interpreted languages, declarative or procedural languages, and can be deployed in any form, including as a stand-alone program or as a module, component, subroutine, object, or other unit suitable for use in a computing environment. A computer program can correspond to a file in a file system. A computer program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatuses can also be implemented as, special purpose logic circuitry, e.g., an FPGA (field programmable gate array) or an ASIC (application specific integrated circuit). Devices suitable for storing computer program instructions and data can include non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

The subject matter described herein can be implemented in a computing system that includes a back end component, e.g., as a data server, or that includes a middleware component, e.g., an application server, or that includes a front end component, e.g., a client computer having a graphical user interface or a web browser through which a user can interact with an implementation of the subject matter described in this specification, or a combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

While operations are depicted in the drawings in a particular order, such operations are not required to be performed in the particular order shown or in sequential order, and all illustrated operations are not required to be performed. Actions described herein can be performed in a different order.

Having now described some illustrative implementations, it is apparent that the foregoing is illustrative and not limiting, having been presented by way of example. In particular, although many of the examples presented herein involve specific combinations of method acts or system elements, those acts and those elements may be combined in other ways to accomplish the same objectives. Acts, elements and features discussed in connection with one implementation are not intended to be excluded from a similar role in other implementations or implementations.

The phraseology and terminology used herein is for the purpose of description and should not be regarded as limiting. The use of “including” “comprising” “having” “containing” “involving” “characterized by” “characterized in that” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

Any references to implementations or elements or acts of the systems and methods herein referred to in the singular may also embrace implementations including a plurality of these elements, and any references in plural to any implementation or element or act herein may also embrace implementations including only a single element. References in the singular or plural form are not intended to limit the presently disclosed systems or methods, their components, acts, or elements to single or plural configurations. References to any act or element being based on any information, act or element may include implementations where the act or element is based at least in part on any information, act, or element.

Any implementation disclosed herein may be combined with any other implementation or embodiment, and references to “an implementation,” “some implementations,” “one implementation” or the like are not necessarily mutually exclusive and are intended to indicate that a particular feature, structure, or characteristic described in connection with the implementation may be included in at least one implementation or embodiment. Such terms as used herein are not necessarily all referring to the same implementation. Any implementation may be combined with any other implementation, inclusively or exclusively, in any manner consistent with the aspects and implementations disclosed herein.

References to “or” may be construed as inclusive so that any terms described using “or” may indicate any of a single, more than one, and all of the described terms. References to at least one of a conjunctive list of terms may be construed as an inclusive OR to indicate any of a single, more than one, and all of the described terms. For example, a reference to “at least one of ‘A’ and ‘B’” can include only ‘A’, only ‘B’, as well as both ‘A’ and ‘B’. Such references used in conjunction with “comprising” or other open terminology can include additional items.

Where technical features in the drawings, detailed description or any claim are followed by reference signs, the reference signs have been included to increase the intelligibility of the drawings, detailed description, and claims. Accordingly, neither the reference signs nor their absence have any limiting effect on the scope of any claim elements.

Modifications of described elements and acts such as variations in sizes, dimensions, structures, shapes and proportions of the various elements, values of parameters, mounting arrangements, use of materials, colors, orientations can occur without materially departing from the teachings and advantages of the subject matter disclosed herein. For example, elements shown as integrally formed can be constructed of multiple parts or elements, the position of elements can be reversed or otherwise varied, and the nature or number of discrete elements or positions can be altered or varied. Other substitutions, modifications, changes and omissions can also be made in the design, operating conditions and arrangement of the disclosed elements and operations without departing from the scope of the present disclosure.

For example, descriptions of positive and negative electrical characteristics may be reversed. Elements described as negative elements can instead be configured as positive elements and elements described as positive elements can instead by configured as negative elements. For example, elements described as having first polarity can instead have a second polarity, and elements described as having a second polarity can instead have a first polarity. Further relative parallel, perpendicular, vertical or other positioning or orientation descriptions include variations within +/−10% or +/−10 degrees of pure vertical, parallel or perpendicular positioning. References to “approximately,” “substantially” or other terms of degree include variations of +/−10% from the given measurement, unit, or range unless explicitly indicated otherwise. Coupled elements can be electrically, mechanically, or physically coupled with one another directly or with intervening elements. Scope of the systems and methods described herein is thus indicated by the appended claims, rather than the foregoing description, and changes that come within the meaning and range of equivalency of the claims are embraced therein.

1. A system, comprising:
one or more processors coupled with a non-transitory memory to:
determine, based on power applied to a vehicle from a source of power of the vehicle, a first emission metric corresponding to an amount of carbon emitted by the power applied to the vehicle over a trip of the vehicle from a first origin location to a second destination location;
determine, based on power consumed by a plurality of components of the vehicle, a corresponding plurality of second emission metrics corresponding to an amount of carbon emitted for each of the components of the vehicle over the trip, wherein the determination uses electrical sensors configured to detect electricity provided to or expended by particular of the plurality of components;
cause a user interface corresponding to the vehicle to present a first indication corresponding to the first emission metric, and a second indication corresponding to the plurality of second emission metrics; and
send a request to a second system in response to input at the user interface, the input comprising a selection of at least one of the second emission metrics, the request indicating a total carbon offset to select based on at least one of the plurality of the second emission metrics.

2. The system of claim 1, comprising:
at least one of the plurality of the components corresponds to a sensor of the component of the vehicle.

3. The system of claim 1, comprising:
the first emission metric corresponding to an estimated amount of carbon emitted by the vehicle over a lifetime of the vehicle, the lifetime of the vehicle including the trip of the vehicle.

4. The system of claim 1, comprising:
the total carbon offset having an aggregate value to offset the amount of carbon emitted by the vehicle.

5. The system of claim 1, comprising:
the one or more processors to:
determine, based on a source metric identifying the source of power, the first emission metric.

6. The system of claim 1, comprising:
the one or more processors to:
determine, based on at least one second emission metric, a third emission metric.

7. The system of claim 1, comprising:
the one or more processors to:
obtain, from the vehicle and via a communication interface compatible with the vehicle, a vehicle metric corresponding to one of the components of the vehicle.

8. The system of claim 1, comprising:
the one or more processors to:
determine, based on the vehicle metric, at least one second emission metric corresponding to at least one component of the vehicle; and
determine, based on the at least one second emission metric, a third emission metric.

9. The system of claim 1, comprising:
the one or more processors to:
obtain, from the vehicle and via a first communication interface compatible with the source of power, an identifier of the source of power;
obtain, based on the identifier and via a second communication interface compatible with a third system, the first emission metric corresponding to the source of power; and
determine, based on the first emission metric, a third emission metric.

10. A method, comprising:
determining, based on power applied to a vehicle from a source of power of the vehicle, a first emission metric corresponding to an amount of carbon emitted by the power applied to the vehicle over a trip of the vehicle from a first origin location to a second destination location;
determining, based on power consumed by a plurality of components of the vehicle, a corresponding plurality of second emission metrics corresponding to an amount of carbon emitted for each of the components of the vehicle over the trip wherein the determination uses electrical sensors configured to detect electricity provided to or expended by particular of the plurality of components;
causing a user interface corresponding to the vehicle to present a first indication corresponding to the first emission metric, and a second indication corresponding to the plurality of second emission metrics; and
sending, in response to input comprising a selection of at least one of the second emission metrics at the user interface, a request to a second system comprising a selection of at least one of the second emission metrics, the request indicating a total carbon offset to select based on at least one of the plurality of the second emission metrics.

11. The method of claim 10, comprising:
at least one of the plurality of the components corresponds to a sensor of the component of the vehicle.

12. The method of claim 10, comprising:
the first emission metric corresponding to an estimated amount of carbon emitted by the vehicle over a lifetime of the vehicle, the lifetime of the vehicle including the trip of the vehicle.

13. The method of claim 10, comprising:
the total carbon offset having an aggregate value to offset the amount of carbon emitted by the vehicle.

14. The method of claim 10, comprising:
determining, based on a source metric identifying the source of power, the first emission metric.

15. The method of claim 10, comprising:
determining, based on at least one second emission metric, a third emission metric.

16. The method of claim 10, comprising:
obtaining, from the vehicle and via a communication interface compatible with the vehicle, a vehicle metric corresponding to one of the components of the vehicle.

17. The method of claim 16, comprising:
determining, based on the vehicle metric, at least one second emission metric corresponding to at least one component of the vehicle; and
determining, based on the at least one second emission metric, a third emission metric.

18. The method of claim 10, comprising:
obtaining, from the vehicle and via a first communication interface compatible with the source of power, an identifier of the source of power;
obtaining, based on the identifier and via a second communication interface compatible with a third system, the first emission metric corresponding to the source of power; and
determining, based on the first emission metric, a third emission metric.

19. A non-transitory computer readable medium, comprising:
one or more instructions stored thereon and executable by a processor to:
determine, by the processor and based on power applied to a vehicle from a source of power of the vehicle, a first emission metric corresponding to an amount of carbon emitted by the power applied to the vehicle over a trip of the vehicle from a first origin location to a second destination location;
determine, by the processor and based on power consumed by a plurality of components of the vehicle, a corresponding plurality of second emission metrics corresponding to an amount of carbon emitted for each of the components of the vehicle over the trip wherein the determination uses electrical sensors configured to detect electricity provided to or expended by particular of the plurality of components;
cause a user interface corresponding to the vehicle to present a first indication corresponding to the first emission metric, and a second indication corresponding to the plurality of second emission metrics; and
send, by the processor and in response to input at the user interface, a request to a second system, the request indicating an asset to select based on at least one of the first emission metric and the second emission metric.

20. The non-transitory computer readable medium of claim 19, comprising:
one or more instructions executable by the processor to:
obtain, from the vehicle and via a communication interface compatible with the vehicle, a vehicle metric corresponding to least one of the plurality of the components of the vehicle;
determine, based on the vehicle metric, the least one of the plurality of second emission metrics corresponding to the at least one component of the vehicle; and
determine, based on the at least one second emission metric, the emission metric.
